(19) United States
(12) Patent Application Publication
(10) Pub. No.: US 2023/0118351 A1
(43) Pub. Date: Apr. 20, 2023

(54) METHOD FOR PRODUCING CLAM ACTIVE PEPTIDE

(71) Applicants: CHENLAND NUTRITIONALS, INC., Pomona, CA (US); QINGDAO CHENLAND PHARMACEUTICAL TECHNOLOGY DEVELOPMENT CO., LTD., Qingdao, Shandong (CN)

(72) Inventors: Tingfeng CAO, Qingdao, Shandong (CN); Fang FAN, Qingdao, Shandong (CN); Shengcan ZOU, Qingdao, Shandong (CN); Guojunnan FENG, Qingdao, Shandong (CN); Jinli LIU, Qingdao, Shandong (CN); Xiu LU, Qingdao, Shandong (CN); Zengliang ZHANG, Qingdao, Shandong (CN)

(73) Assignees: CHENLAND NUTRITIONALS, INC., Pomona, CA (US); QINGDAO CHENLAND PHARMACEUTICAL TECHNOLOGY DEVELOPMENT CO., LTD., Qingdao, Shandong (CN)

(21) Appl. No.: 16/978,674

(22) PCT Filed: Feb. 20, 2020
(86) PCT No.: PCT/CN2020/076042

§ 371 Date: Sep. 4, 2020

(30) Foreign Application Priority Data
Jan. 16, 2020 (CN) 202010047257.8

(51) Int. Cl. C07K 1/34; C12P 21/06

(52) U.S. Cl. CPC C07K 1/34; C12P 21/06

(57) ABSTRACT

This application belongs to the field of biotechnology and discloses a method for producing a clam active peptide. The method for producing a clam active peptide comprises cleaning fresh clam meat with water, adding water and homogenizing with a colloid mill to prepare a clam meat slurry; adding water and complex protease for enzymolysis of the clam meat slurry, and heating to inactivate enzyme after the enzymolysis; centrifuging to collect an enzymatic hydrolyzate, capturing the enzymatic hydrolyzate having a molecular weight of lower than 2 KDa through microfiltration-ultrafiltration-nanofiltration membrane filtration, and drying to obtain the clam active peptide. The present disclosure produces a clam active peptide having pure color, outstanding taste, and blood pressure lowering function which is easily absorbed by human body using fresh clam meat as raw material, adopting a complex enzyme-membrane coupling technology through processing techniques such as enzymolysis, membrane separation purification and drying.

This application claims the priority of the Chinese patent application No. 202010047257.8 filed on Jan. 16, 2020, and titled with “Method for producing clam active peptide”, and the disclosure of which is hereby incorporated by reference.

### FIELD

The present invention belongs to the field of biotechnology, and in particular relates to a method for producing clam active peptide, in particular to a method suitable for large-scale industrial production of clam active peptide.

### BACKGROUND

Clams are one of the four major cultured shellfish in China and are rich in resources. Clams are delicious in meat, having nutritional characteristics of high protein, high vitamins, low fat, and containing more than ten kinds of amino acids and minerals necessary for the human body. Clam is a bivalve shellfish marine product with dual use of food and medicine, and has become a recognized healthy food with high nutrition and low costs. In China, clams are mainly present as fresh food and dried product, and problems such as low processing level and few product types in this industry are becoming increasingly prominent. With the increasing demand of consumers for high-quality marine foods, the backward traditional processing technology can no longer meet human needs. Therefore, how to realize the deep processing and high-value utilization of clams and develop more and better functional clam products is a huge opportunity and severe challenge in the clam industry.

With the continuous deepening of the utilization of marine resources, small peptides of marine organism derived from bio-enzymolysis technology have been widely concerned due to their advantages of small molecular weight, high biological potency, good physiological activity, well stability, safety and portability. Up to now, there are substances such as sea cucumber peptides, oyster peptides, abalone peptides, collagen peptides sold in large quantities in the domestic market, while no sales of clam peptides and related clam extract products have been found.

Studies on the extraction of active substances from marine organisms have long been reported. Modern studies have proved that marine biological extracts have effects of improving immunity, anti-tumor, lowering blood pressure, anti-bacteria, inhibiting the formation of micronuclei in cells, and resisting atherosclerosis. Therefore, in recent years, studies on the preparation of marine bioactive peptides and the mechanism of the active substances thereof have become a hot spot. Currently, a large number of functionally active peptides have been obtained from proteins of terrestrial organisms and marine organisms such as oysters, sea cucumbers, and marine fish by enzymolysis. However, there are relatively few reports on the polypeptides obtained by enzymolysis of clam proteins. Enzyme preparations used in the existing reports on enzymolysis of clam proteins include pepsin, trypsin, papain, neutral protease, alkaline protease and animal hydrolyzed protease, and the like. However, there is no report on the study of complex protease for enzymolysis of proteins of Hongdao clam meat to extract polypeptides.

In the patent titled “A method of extracting a clam active peptide” (Chinese patent application No. 20161117416.8), polypeptides are obtained by two methods of flocculation-centrifugation-dextran gel column separation and enzymolysis-dextran gel column separation. However, the process of gel column separation is difficult to apply to large-scale production. In the patent titled “A method of extracting a clam peptide” (Chinese patent application No. 20161117416.8), an enzymolysis method comprising pH adjustment with sodium hydroxide, enzymolysis, extraction, enzyme inactivation, centrifugation, filtration and adsorption, nanofiltration separation and concentration as well as drying in sequence is performed. However, the reaction process is complicated, which has high costs for large-scale production and chemicals such as sodium hydroxide are added in the process.

### SUMMARY

In view of this, the purpose of the present disclosure is to provide a production method suitable for large-scale industrial production of clam active peptide, to overcome the shortcomings of the prior art.

To achieve the purpose of the present disclosure, the following technical solutions are adopted in the present disclosure:

A method for producing a clam active peptide, comprising cleaning fresh clam meat with water, adding water and homogenizing with a colloid mill to prepare a clam meat slurry; adding water and complex protease for enzymolysis of the clam meat slurry, and heating to inactivate enzyme after the enzymolysis; centrifuging to collect an enzymatic hydrolyzate, capturing the enzymatic hydrolyzate having a molecular weight of lower than 2 KDa through microfiltration-ultrafiltration-nanofiltration membrane filtration, and drying to obtain the clam active peptide.

The present disclosure produces a clam active peptide having pure color, outstanding taste, and blood pressure lowering function which is easily absorbed by human body using fresh clam meat as raw material, adopting a complex enzyme-membrane coupling technology through processing techniques such as enzymolysis, membrane separation and purification as well as drying, in order to achieve high-value utilization of clam meat.

In the present disclosure, in the production method, the complex protease consists of neutral protease, alkaline protease, and flavor protease, and the addition ratio of neutral protease: alkaline protease: flavor protease is 2:1:1.

In the present disclosure, in the production method, the complex protease is added in an amount of 0.1%-0.3% by weight of the clam meat slurry; during the enzymolysis process, continuous stirring is performed to make full use of the complex protease and ensure complete enzymolysis.

In the present disclosure, in the production method, weight ratio of the clam meat to water during enzymolysis is 1: 1-1:3.

In the present disclosure, in the production method, the clam meat is Hongdao clam meat.

In the present disclosure, in the production method, the cleaning is performed using deionized water.

In the present disclosure, in the production method, the homogenization is performed according to the weight ratio of clam meat: water=1:1.

In the present disclosure, in the production method, the added water is deionized water; during the homogenization with a colloid mill, the gaps of particles in the colloid mill are controlled at 0-5 to ensure that the particle size of the clam meat is small and uniform.

In the present disclosure, in the production method, the enzymolysis is performed under natural pH conditions at 50-60° C. for 4-6 hours.

In the present disclosure, in the production method, the heating to inactivate enzyme is performed by heating the enzymatic hydrolyzate to 85° C. for 10 minutes.

In the present disclosure, in the production method, the centrifugation is performed by cooling the enzymatic hydrolyzate to below 40° C., filtering through a 200-300 mesh sieve, and then centrifuging at 16000 r/min.

In the present disclosure, in the production method, the drying is performed by spray drying of the filtrate after membrane filtration in a drying tower. The spray drying is performed at a temperature of 150-180° C., which can realize instantaneous drying into powder.

The above-mentioned industrial production method is performed under mild conditions and is easy to control. The obtained clam active peptide has a pure flavor and a small molecular weight, is easily absorbed and has a higher quality. Therefore, the present disclosure also provides the clam active peptide prepared by the method.

Compared with the prior art, the present disclosure has at least one of the following beneficial effects:
(1) In the prior art, the method for extracting a clam active peptide generally have the problems of complex processes, high production costs, and long cycles, and are generally suitable for laboratory preparation and are not suitable for industrial production. The method of the present disclosure is characterized by simple processes, mild conditions, short cycles, without addition of any inorganic or organic solvents, low energy consumption and high yield, which is more suitable for industrial production.
(2) In the prior art, single enzymes such as papain (endonuclease), neutral protease (endonuclease), and flavor protease (exonuclease) are mostly used as enzyme preparations, and the enzymatic hydrolyzate prepared therefrom is characterized by a low protein recovery rate, a large average relative molecular mass of the active clam peptide, a small proportion of the protein hydrolysate with a relative molecular mass of less than 1000 U, and a low polypeptide content. The present disclosure prepares a calm enzymatic hydrolyzate using complex protease to hydrolyze proteins in the clam meat through a complex enzyme-membrane coupling technology, and the obtained calm enzymatic hydrolyzate has a high protein recovery rate (that is, high contents of effective components in the enzymatic hydrolyzate), which is up to 90%, high yield, greater than 90% of the protein hydrolysate with a molecular weight of less than 1000 U, and greater than 80% of the content of polypeptides, and the product quality pass rate is high. The enzymatic hydrolyzate obtained from enzymolysis of calm meat with complex protease is then subjected to a centrifugation-membrane filtration (microfiltration-ultrafiltration-nanofiltration)-drying technology to obtain enzymatic hydrolysate, which does not require for decolorization, deodorization and further purification and concentration. The process is simple and has high production efficiency and low costs while there is no need for decolorization and deodorization using activated carbon, activated carbon fiber, and the like, thus avoiding the generation of a large amount of solid waste, which is more suitable for industrial production.
(3) The clam active peptide produced in the present disclosure is mainly tetrapeptide to hexapeptide, which has a high ACE inhibitory activity with an ACE inhibitory rate up to 85%, and a high activity of lowering blood pressure.
(4) The clam active peptide produced in the present disclosure is characterized by pure color of almost white, excellent flavor, outstanding taste, high sensory evaluation with no fishy smell and other odor, and is more popular with consumers. Moreover, the clam active peptide has a small molecular weight and is easy to be absorbed by human body, and it is further rich in nutrients such as free amino acids, taurine and selenium, which can truly realize a high-value utilization of the clam meat.

### BRIEF DESCRIPTION OF DRAWINGS

In order to more clearly illustrate the technical solutions in the examples of the present invention or in the prior art, the drawings used in the examples or the prior art will be briefly introduced below.

FIG. 1 shows the protein recovery rate of clam meat enzymolysis by different proteases in Example 1;

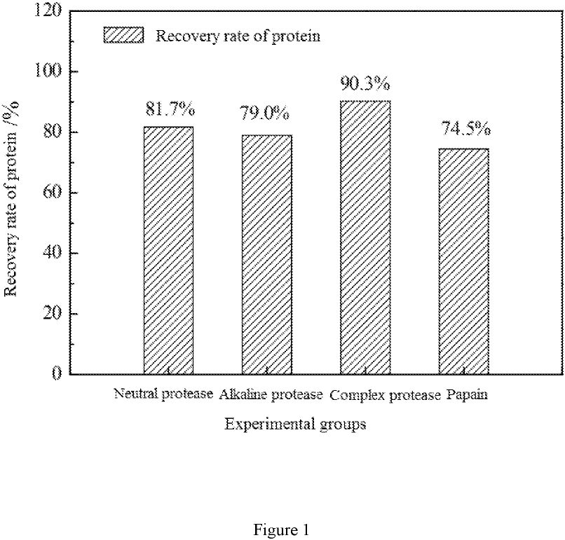

FIG. 2 shows the content of the polypeptide in the enzymatic hydrolyzate and the proportion of the hydrolysate having a relative molecular mass of less than 1000 U under different enzymolysis conditions in Example 1;

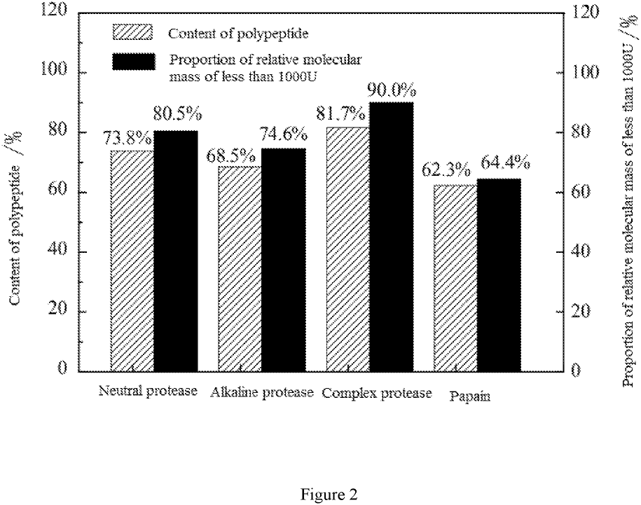

FIG. 3 shows a chromatogram of the clam active peptide sample prepared in Example 2.

### DETAILED DESCRIPTION

The present disclosure discloses a method for producing a clam active peptide. Those skilled in the art can learn from the disclosure and appropriately improve the process parameters. In particular, it should be noted that all similar substitutions and modifications will be obvious to those skilled in the art, which are all considered to be incorporated in the present disclosure. The methods and products of the present disclosure have been described in preferred embodiments, and it is obvious for relevant persons to modify or appropriately change and combine the methods described herein without departing from the content, spirit, and scope of the present disclosure to implement and apply the technology of the disclosure.

To achieve the purpose of the present disclosure, the following technical solutions are adopted in the present disclosure:

A method for producing a clam active peptide, comprising cleaning fresh clam meat with water, adding water and homogenizing with a colloid mill to prepare a clam meat slurry; adding water and complex protease for enzymolysis of the clam meat slurry, and heating to inactivate enzyme after the enzymolysis; centrifuging to collect an enzymatic hydrolyzate, capturing the enzymatic hydrolyzate having a molecular weight of lower than 2 KDa through microfiltration-ultrafiltration-nanofiltration membrane filtration, and drying to obtain the clam active peptide.

In some embodiments, in the production method, the clam meat is Hongdao clam meat. Fresh Hongdao clam meat is used as a substrate for enzymolysis, which is reliable and safe in source, has a high nutritional value and extremely low content of harmful substances such as heavy metals.

In the present disclosure, in the production method, fresh clam meat is firstly cleaned with water for pre-treatment to remove the impurities on the surface of the clam meat. Fresh clam meat itself is rich in nutrients such as free amino acids, vitamins, zinc, and selenium. If the cleaning in the pre-treatment is improper, a large number of nutrients will be lost. The cleaning in the present disclosure is performed by using deionized water then draining slightly. The surface of the clam meat is cleaned simply with deionized water to remove the impurities and then drained the cleaned clam meat slightly. In the present disclosure, cleaning, draining and drying are not performed excessively, ensuring that the moisture and nutrient components in the clam meat are not lost. The pre-treatment process in the production method of the present disclosure is simple, convenient and easy to operate.

In the present disclosure, in the production method, weight ratio of the clam meat to water is 1:1 during the homogenizing with a colloid mill. In some embodiments, the water is deionized water.

Further, in the present disclosure, during the homogenization with a colloid mill, the gaps of particles in the colloid mill are controlled at 0-5 to ensure that the particle size of the clam meat is small and uniform, such that the prepared clam meat slurry is delicate and easy to be hydrolyzed and utilized by complex protease.

Endonucleases are a type of nucleic acid hydrolase that can hydrolyze phosphodiester bonds from the middle of the protein molecules, thereby cutting double-stranded proteins; while exonucleases can only cut from one end of the protein molecules. The production method in the present disclosure uses complex protease for enzymolysis. The complex protease consists of neutral protease, alkaline protease, and flavor protease, and is a complex enzyme of a variety of endonucleases and exonucleases. The complex protease has more enzyme cutting sites, which can have a more thorough enzymolysis to the proteins in clam meat, thereby improving the recovery rate of proteins in the clam meat, reducing the average molecular weight of the enzymatic hydrolyzate, and facilitating the subsequent membrane filtration process. Enzymolysis using the composite protease of the present disclosure can not only ensure the quality of clam active peptide products, but also greatly improve the product yield and increase economic benefits.

In the present disclosure, the addition ratio of neutral protease: alkaline protease: flavor protease is 2:1:1.

In the present disclosure, in the production method, during enzymolysis the complex protease is added in an amount of 0.1%-0.3% by weight of the clam slurry. In some embodiments, the complex protease is added in an amount of 0.13% by weight of the clam slurry. In some embodiments, the complex protease is added in an amount of 0.2% by weight of the clam slurry. In some embodiments, the complex protease is added in an amount of 0.3% by weight of the clam slurry.

In the present disclosure, in the production method, the weight ratio of the clam meat to water during enzymolysis is 1:1-1:3. In some embodiments, the water added during enzymolysis is deionized water.

In the present disclosure, in the production method, the enzymolysis is performed under natural pH conditions at 50-60° C. for 4-6 hours. In some embodiments, the enzymolysis specifically comprises adding the clam meat slurry obtained by homogenization as a substrate to an enzymolysis tank, adding deionized water, heating the enzymolysis tank to 50-60° C., adding complex protease, to perform enzymolysis under natural pH conditions for 4-6 h. The enzymolysis process of the present disclosure is performed at natural pH without addition of chemical reagents such as hydrochloric acid and sodium hydroxide to adjust the pH of the clam slurry. The process is simple and the operating conditions are mild and easy to control. The process needs no addition of any chemical reagents and has low energy consumption and cost.

Further, during the enzymolysis process, continuous stirring is performed to make full use of the complex protease and ensure complete enzymolysis.

The production method of the present disclosure includes heating to inactivate enzyme after the enzymolysis. In the present disclosure, the heating to inactivate enzyme is performed by heating the enzymatic hydrolyzate to 85° C. for 10 minutes. The inactivation temperature and tim should not exceed the value mentioned above, which not only ensures that the enzymatic hydrolyzate is fully inactivated, but also avoids the occurrence of the Maillard reaction of the enzymatic hydrolyzate itself at high temperature, causing decrease of the nutrients in the enzymatic hydrolyzate and darkening of the color of the enzymatic hydrolyzate and ultimately affecting the quality of clam active peptides.

The production method of the present disclosure comprises centrifuging to collect an enzymatic hydrolyzate after enzyme inactivation. In some embodiments, the centrifugation is performed by cooling the enzymatic hydrolyzate to below 40° C., filtering through a 200-300 mesh sieve, and then centrifuging at 16000 r/min.

Further, the enzymatic hydrolyzate after centrifugation is subjected to membrane filtration to capture the enzymatic hydrolyzate having a molecular weight of less than 2 KDa. The membrane filtration is specifically microfiltration-ultrafiltration-nanofiltration membrane filtration.

In the present disclosure, in the production method, the drying is performed by spray drying of the filtrate after membrane filtration in a drying tower. The spray drying is performed at 150-180° C., which can realize instantaneous drying into powder.

Compared with processes such as centrifugation-plate-frame pressure filtration -ultrafiltration-nanofiltration-negative pressure concentration-spray drying, centrifugation-ultrafiltration-reduced pressure concentration-spray drying, centrifugation-resin filtration and adsorption-nanofiltration-concentration-spray drying in the prior art, in the present disclosure, a process of enzymatic hydrolyzate centrifugation-membrane filtration-spray drying is adopted, which is simple, easy to operate, and has low costs, and is more suitable for industrial production. The clam active peptide product obtained in the method of the present disclosure is characterized by pure color, good flavor and high quality, which needs no operation processes such as decolorization and deodorization or further purification and concentration.

The production method in the present disclosure is performed under mild conditions and easy to control. The obtained clam active peptide is mainly tetrapeptide to hexapeptide, which is characterized by pure flavor, and small molecular weight, and has the function of lowering blood pressure, is easy to be absorbed and has higher quality. Therefore, the present disclosure also provides the clam active peptide prepared by the method.

Compared with the prior art, the present disclosure has at least one of the following beneficial effects:
(1) In the prior art, the method for extracting a clam active peptide generally have the problems of complex processes, high production costs, and long cycles, and are generally suitable for laboratory preparation and are not suitable for industrial production. The method of the present disclosure is characterized by simple processes, mild conditions, short cycles, without addition of any inorganic or organic solvents, low energy consumption and high yield, which is more suitable for industrial production.
(2) In the prior art, single enzymes such as papain (endonuclease), neutral protease (endonuclease), and flavor protease (exonuclease) are mostly used as enzyme preparations, and the enzymatic hydrolyzate prepared therefrom is characterized by a low protein recovery rate, a large average relative molecular mass of the active clam peptide, a small proportion of the protein hydrolysate with a relative molecular mass of less than 1000 U, and a low polypeptide content. The present disclosure prepares a calm enzymatic hydrolyzate using complex protease to hydrolyze proteins in the clam meat through a complex enzyme-membrane coupling technology, and the obtained calm enzymatic hydrolyzate has a high protein recovery rate (that is, high contents of effective components in the enzymatic hydrolyzate), which is up to 90%, high yield, greater than 90% of the protein hydrolysate with a molecular weight of less than 1000 U, and greater than 80% of the content of polypeptides, and the product quality pass rate is high. The enzymatic hydrolyzate obtained from enzymolysis of calm meat with complex protease is then subjected to a centrifugation-membrane filtration (microfiltration-ultrafiltration-nanofiltration)-drying technology to obtain enzymatic hydrolysate, which does not require for decolorization, deodorization and further purification and concentration. The process is simple and has high production efficiency and low costs while there is no need for decolorization and deodorization using activated carbon, activated carbon fiber, and the like, thus avoiding the generation of a large amount of solid waste, which is more suitable for industrial production.
(3) The clam active peptide produced in the present disclosure is mainly tetrapeptide to hexapeptide, which has a high ACE inhibitory activity with an ACE inhibitory rate up to 85%, and a high activity of lowering blood pressure.
(4) The clam active peptide produced in the present disclosure is characterized by pure color of almost white, excellent flavor, outstanding taste, high sensory evaluation with no fishy smell and other odor, and is more popular with consumers. Moreover, the clam active peptide has a small molecular weight and is easy to be absorbed by human body, and it is further rich in nutrients such as free amino acids, taurine and selenium, which can truly realize a high-value utilization of the clam meat.

In order to further understand the present disclosure, the technical solutions in the embodiments of the present disclosure will be described clearly and completely in conjunction with the examples of the present disclosure. It is obvious that the described examples are only a part but not all the embodiments of the present disclosure. Based on the examples of the present disclosure, all other examples obtained by a person of ordinary skill in the art without creative work fall within the protection scope of the present disclosure.

Unless otherwise specified, the reagents involved in the examples of the present invention are all commercially available products and can be purchased through commercial channels.

Among them, the method of determining the ACE inhibitory rate is specifically as follows: 100 µL of 5.0 mmol/L N-Hippuryl-His-Leu hydrate (HHL) solution was mixed with 30 µL of clam peptide solution (ACEI), and the mixture was placed in a water bath at 37° C. for 10 min, then 10 µL of 0.1 U/mL ACE enzyme solution was added. The mixture was mixed and the reaction was continued in the water bath at 37° C. for 30 min. 250 µL of 1 mol/L HCl was then added to the reaction system to terminate the reaction, and then 1.2 mL of frozen ethyl acetate was added to extract the generated hippuric acid. After vortex and shake mixing, centrifugation was performed at 3500 r/min for 5 min. 1.0 mL of the ethyl acetate layer was sucked and dried in an oven at 90° C. for 1 hour. After cooling, 4 mL of distilled water was added to dissolve sufficiently. After vortex mixing, the absorbance OD228 was measured at the wavelength of 228 nm. The parallel control group had the same operating steps with that of the experimental group except that 250 µL of 1 mol/L HCl was added before the reaction to terminate the reaction. The measurement was repeated for 3 times to get averaged value of the results. The specific operation steps were shown in Table 1:

TABLE 1

Determination of ACE inhibitory rate by ultraviolet spectrophotometer

| | | | |
|---|---|---|---|
| Reagents | Experimental group (a) | Control group (b) | Blank group (c) |
| 5.0 mmol·L⁻¹HHL/(µL) | 100 | 100 | 100 |
| 1 mol·L⁻¹HCl/(µL) | 0 | 0 | 250 |
| ACE I(µL) | 30 | 0 | 0 |
| Mixing and water-bathing at 37° C. for 10 min | | | |
| 0.1 U/mL ACE | 10 | 10 | 10 |
| Mixing and water-bathing at 37° C. for 30 min | | | |
| 1 mol·L⁻¹HCl/(µL) | 250 | 250 | 0 |
| ACE I/(µL) | 0 | 30 | 30 |
| Ethyl acetate /(µL) | 1.2 | 1.2 | 1.2 |
| Mixing and shaking for 2 min, centrifugating at 3500 r/min for 5 min, and standing for 2 min Sucking 1.0 mL of the ethyl acetate layer, drying at 90° C. for 1h | | | |
| Distilled water /(mL) | 4 | 4 | 4 |
| OD₂₂₈ | Aₐ | A_{b} | A_{c} |

The calculation formula is:

$$\text{ACE inhibition rate} \text{=} \frac{{A}_{b} - {A}_{a}}{{A}_{b} - {A}_{a}} \times 100 \%$$

In the formula: Aₐ—absorbance value of the reaction with HHL when both ACE and its inhibitor are present in the reaction; A_{b}—absorbance value of the reaction of ACE enzyme and HHL when the ACE inhibitor is not included in the reaction; A_{c}—absorbance value of the blank reaction of ACE and HHL.

The method of determining the protein recovery rate is as follows: according to the first method named Kjeldahl method in GB5009.5 “Determination of Protein in Food Safety National Standards”, the protein content a1 in raw clam meat and the protein content b1 in clam active peptide powder were determined respectively. The clam active peptide was produced according to the method of the present disclosure, and the feeding amount A₁ of fresh clam meat and the receiving amount B₁ of clam active peptide powder were recorded, and the formula for calculating the protein recovery rate X of the clam active peptide powder was:

$$\begin{matrix}\text{Protein recovery rate x/\%} \text{=} \frac{\text{Total protein content in clam peptide powder}}{\text{Total protein content in clam meat}} \\ \times 100 = \frac{{b}_{1} \times {B}_{1}}{{a}_{1} \times {A}_{1}} \times 100 ,\end{matrix}$$

### Example 1: Comparison of Enzymolysis Effects of Different Proteases

Raw meat: Hongdao clam meat.

Test enzymes: neutral protease, alkaline protease, papain, complex protease.

Process flow for enzymolysis: frozen Hongdao clam meat was thawed, and then deionized water was added according to a weight ratio of clam meat: water = 1:1 for homogenization; enzymolysis was performed at a temperature of 50° C. and natural pH using neutral protease, alkaline protease, papain, and complex protease (the formulation was neutral protease: alkaline protease: flavor protease=2:1:1) with addition of the enzyme in an amount of 0.13% of the clam slurry; enzymolysis was performed separately at a constant temperature for 6 h, and the enzymes were inactivated at 85° C. for 10 min. The mixture was centrifuged at 4000 r/min for 30 min to obtain the supernatant.

TABLE 2

Test results of the products obtained by enzymolysis

| Test items | | Protein recovery rate in enzymolysis supernatant/% | Polypeptide content/% | Proportion of the protein hydrolysate with relative molecular mass of less than 1000 U/% |
|---|---|---|---|---|
| Test results | Neutral protease | 81.7 | 73.8 | 80.5 |
| | Alkaline protease | 79.0 | 68.5 | 74.6 |
| | Papain | 74.5 | 62.3 | 64.4 |
| | Complex protease | 90.3 | 81.7 | 91.8 |

It can be seen from the results in Table 2 that among the four selected proteolytic enzymes, the protein recovery rate, polypeptide content, and proportion of the protein hydrolysate with relative molecular mass of less than 1000 U of the clam enzymatic hydrolyzate hydrolyzed by complex protease are all larger than those of papain, neutral protease, and alkaline protease, and the average relative molecular mass of the clam enzymatic hydrolyzate hydrolyzed by the complex protease is the smallest. Therefore, the hydrolase is preferably complex protease.

### Example 2: Industrial Method for Producing the Clam Active Peptide According to The Present Disclosure

Raw material meat: Hongdao clam meat.

Test enzyme: complex protease.

Process flow for enzymolysis: Frozen Hongdao clam meat was thawed, and then deionized water was added according to the weight ratio of clam meat: water = 1:1 for homogenization. A certain amount of deionized water was added to the clam meat slurry such that the final weight ratio of clam meat: water=1:2; enzymolysis was performed at a temperature of 50° C. and natural pH using complex protease (the formulation was neutral protease: alkaline protease: flavor protease=2:1:1) with addition of the enzyme in an amount of 0.13% of the clam slurry; enzymolysis was performed separately at the constant temperature for 4 h, and the enzymes were inactivated at 85° C. for 10 min. The mixture was centrifuged at 16000 r/min to obtain a supernatant. The supernatant was performed membrane filtration and spray drying to obtain the clam active peptide.

### Example 3: Industrial Method for Producing the Clam Active Peptide According to The Present Disclosure

Raw material meat: Hongdao clam meat.

Test enzyme: complex protease.

Process flow for enzymolysis: Frozen Hongdao clam meat was thawed, and then deionized water was added according to the weight ratio of clam meat: water = 1:1 for homogenization. A certain amount of deionized water was added to the clam meat slurry such that the final weight ratio of clam meat: water=1:3; enzymolysis was performed at a temperature of 50° C. and natural pH using complex protease (the formulation was neutral protease: alkaline protease: flavor protease=2:1:1) with addition of the enzyme in an amount of 0.13% of the clam slurry; enzymolysis was performed separately at the constant temperature for 4h, and the enzymes were inactivated at 85° C. for 10 min. The mixture was centrifuged at 16000 r/min to obtain a supernatant. The supernatant was performed membrane filtration and spray drying to obtain the clam active peptide.

### Example 4: Industrial Method for Producing the Clam Active Peptide According to The Present Disclosure

Raw material meat: Hongdao clam meat.

Test enzyme: complex protease.

Process flow for enzymolysis: Frozen Hongdao clam meat was thawed, and then deionized water was added according to the weight ratio of clam meat: water = 1:1 for homogenization. A certain amount of deionized water was added to the clam meat slurry such that the final weight ratio of clam meat: water=1:2; enzymolysis was performed at a temperature of 60° C. and natural pH using complex protease (the formulation was neutral protease: alkaline protease: flavor protease=2:1:1) with addition of the enzyme in an amount of 0.2% of the clam slurry; enzymolysis was performed separately at the constant temperature for 4 h, and the enzymes were inactivated at 85° C. for 10 min. The mixture was centrifuged at 16000 r/min to obtain a supernatant. The supernatant was performed membrane filtration and spray drying to obtain the clam active peptide.

### Example 5: Industrial Method for Producing the Clam Active Peptide According to The Present Disclosure

Raw meat: Hongdao clam meat.

Test enzyme: complex protease.

Process flow for enzymolysis: Frozen Hongdao clam meat was thawed, and then deionized water was added according to the weight ratio of clam meat: water = 1:1 for homogenization. A certain amount of deionized water was added to the clam meat slurry such that the final weight ratio of clam meat: water=1:2; enzymolysis was performed at a temperature of 50° C. and natural pH using complex protease (the formulation was neutral protease: alkaline protease: flavor protease=2:1:1) with addition of the enzyme in an amount of 0.3% of the clam slurry; enzymolysis was performed separately at the constant temperature for 6 h, and the enzymes were inactivated at 85° C. for 10 min. The mixture was centrifuged at 16000 r/min to obtain a supernatant. The supernatant was performed membrane filtration and spray drying to obtain the clam active peptide.

### Test Example

The clam active peptide prepared in each example was tested, and the results were shown in Table 3.

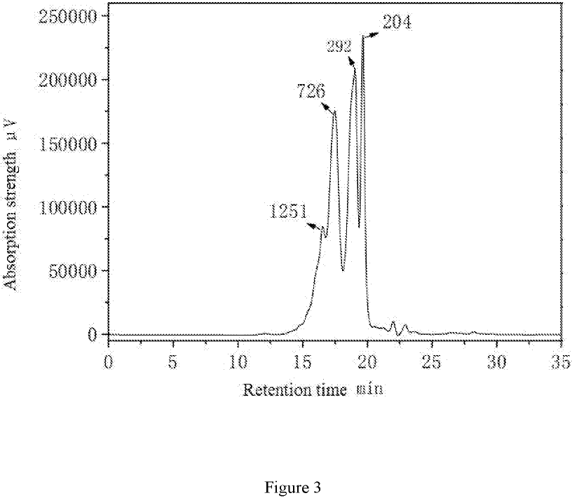

The clam active peptide prepared in each example was analyzed by liquid chromatography, in which the results of the clam active peptide prepared in Example 2 were shown in FIG. 3.

TABLE 3

Test results of the clam active peptide

| Items | Test data | | | | Test method |
|---|---|---|---|---|---|
| | Example 2 | Example 3 | Example 4 | Example 5 | |
| ACE inhibitory rate/% | 86.8 | 85.1 | 86.3 | 85.8 | UV spectrophotometry |
| Protein content/ ( g/100 g ) | 84.2 | 82.2 | 85.4 | 85.6 | The first method in GB5009.5 |
| Protein recovery rate/% | 90.8 | 91.2 | 91.8 | 92.3 | / |
| Polyeptide content/% | 82.1 | 80.5 | 83.3 | 83.5 | GB/T22729 |
| Proportion of relative molecular mass of <1000 U/% | 90.2 | 91.9 | 91.0 | 91.8 | Appendix A in GB/T22729 |
| Proportion of relative molecular mass of 1000-2000 U/% | 7.7 | 6.6 | 7.1 | 6.5 | |
| Proportion of relative molecular mass of 2000-5000 U/% | 1.9 | 1.4 | 1.7 | 1.6 | |
| Moisture/(g/100 g) | ≤8.0 | ≤8.0 | ≤8.0 | ≤8.0 | GB5009.3 |
| Ash/(g/100 g) | ≤7.0 | ≤7.0 | ≤7.0 | ≤7.0 | GB5009.4 |
| Inorganic arsenic/(mg/kg) | ≤0.5 | ≤0.5 | ≤0.5 | ≤0.5 | GB5009.11 |
| Methyl mercury/(mg/kg) | ≤0.5 | ≤0.5 | ≤0.5 | ≤0.5 | GB5009.17 |
| Plumbum/(mg/kg) | ≤1.0 | ≤1.0 | ≤1.0 | ≤1.0 | GB5009.123 |
| Chromium/(mg/kg) | ≤2.0 | ≤2.0 | ≤2.0 | ≤2.0 | GB5009.123 |
| Total colonies/(CFU/g) | <10 | <10 | <10 | <10 | GB4789.2 |
| E.coli/(CFU/g) | <10 | <10 | <10 | <10 | The plate count method in GB4789.3 |
| Salmonella | Not detected | Not detected | Not detected | Not detected | GB4789.4 |
| Vibrio parahemolyticus/(MPN/g) | Not detected | Not detected | Not detected | Not detected | GB4789.7 |
| Staphylococcus aureus (CFU/g) | Not detected | Not detected | Not detected | Not detected | The second method in GB4789.10 |

It can be seen from the results in Table 3 that in the clam active peptide product, the protein hydrolysate with a relative molecular mass ≤2000 U accounts for above 97%, and the protein hydrolysate with a relative molecular mass <1000 U accounts for above 90%, and the content of small molecule peptides is higher, which is easier to be absorbed. The heavy metal pollutants index and microorganisms index of the clam active peptide meet national standards; the clam active peptide has a good ACE inhibitory activity with the ACE inhibitory rate of greater than 85%. Therefore, the clam active peptide prepared in the disclosure has strong blood pressure lowering function. In addition, the clam active peptide product has pure color, and has no fishy smell or other odor.

The test data of the clam active peptide product prepared by the production method of the present disclosure all meet the standard requirements, and the quality thereof is reliable. Moreover, the relative molecular mass of the clam active peptide product prepared by the production method of the present disclosure is less than 1300 U. The clam active peptide is mainly small molecular peptide such as tetrapeptide, pentapeptide, and hexapeptide, which has a small molecular weight and is easy to be absorbed.

The above is only preferred embodiments of the present disclosure, and is not intended to limit the present disclosure in other forms. It should be pointed out that any person skilled in the art may make improvement and refinement the technical contents disclosed above into equivalent examples with equivalent changes. However, any simple modifications and equivalent changes made to the above examples according to the technical essence of the present disclosure without departing from the content of the present disclosure still fall within the protection scope of the present disclosure.

1. A method for producing a clam active peptide, comprising
cleaning fresh clam meat with water, adding water and homogenizing with a colloid mill to prepare a clam meat slurry; adding water and complex protease for enzymolysis of the clam meat slurry, and heating to inactivate enzyme after the enzymolysis; centrifuging to collect an enzymatic hydrolyzate, capturing the enzymatic hydrolyzate having a molecular weight of lower than 2 KDa through microfiltration-ultrafiltration-nanofiltration membrane filtration, and drying to obtain the clam active peptide.

2. The method according to claim 1, wherein the complex protease consists of neutral protease, alkaline protease, and flavor protease, and the addition ratio of neutral protease: alkaline protease: flavor protease is 2:1:1.

3. The method according to claim 1, wherein the complex protease is added in an amount of 0.1%-0.3% by weight of the clam meat slurry; during the enzymolysis process, continuous stirring is performed to make full use of the complex protease and ensure complete enzymolysis.

4. The method according to claim 1, wherein weight ratio of the clam meat to water during enzymolysis is 1:1-1:3.

5. The method according to claim 1, wherein the clam meat is Hongdao clam meat; the cleaning is performed using deionized water; and the homogenization is performed according to the weight ratio of clam meat: water=1:1; the added water is deionized water; during the homogenization with a colloid mill, the gaps of particles in the colloid mill are controlled at 0-5 to ensure that the particle size of the clam meat is small and uniform.

6. The method according to claim 1, wherein the enzymolysis is performed under natural pH conditions at 50-60° C. for 4-6 hours.

7. The method according to claim 1, wherein the heating to inactivate enzyme is performed by heating the enzymatic hydrolyzate to 85° C. for 10 minutes.

8. The method according to claim 1, wherein the centrifugation is performed by cooling the enzymatic hydrolyzate to below 40° C., filtering through a 200-300 mesh sieve, and then centrifuging at 16000 r/min.

9. The method according to claim 1, wherein the drying is performed by spray drying of the filtrate after membrane filtration in a drying tower.

10. Clam active peptide prepared by the method of claim 1.
